(19) United States
(12) Patent Application Publication
(10) Pub. No.: US 2019/0380728 A1
(43) Pub. Date: Dec. 19, 2019

(54) ULTRASONIC PROBE

(71) Applicant: A Hoyos LLC, Miami, FL (US)

(72) Inventor: Alfredo Ernesto Hoyos Ariza, Miami, FL (US)

(21) Appl. No.: 16/012,254

(22) Filed: Jun. 19, 2018

(51) Int. Cl. A61B 17/22; A61B 17/32

(52) U.S. Cl. CPC A61B 17/22012; A61B 2017/320073; A61B 2017/320069; A61B 17/320068

(57) ABSTRACT

A probe attachable to an ultrasonic generator and structured to conduct ultrasonic energy to a surgical site. The probe comprises an elongated body with a sidewall that extends between a proximal end and a distal end. The probe further comprises one projection(s) that extends outwardly from the sidewall and that is structured to remove fat tissue. The body comprises a distal tip disposed between the distal end and the projection(s). The distal tip comprises a substantially conical configuration that diverges continuously from the distal end to the projection(s). The distal tip comprises an apex that is disposed coincident to the distal end, and that comprises a substantially rounded, blunt configuration. The projection(s) comprises a curved configuration and extends transversely, and at least partially, around the sidewall. The distal tip and the projection(s) are structured and dimensioned for movable and removable positioning within the surgical site.

### BACKGROUND OF THE INVENTION

### Field of the Invention

The invention relates to ultrasonic devices to assist with emulsification and removal of fat tissue during surgical procedures.

### Description of the Related Art

Ultrasound-based techniques for reshaping the body are known to exist. These techniques typically require a small dose of an anesthetic for insertion of a probe that can remove fat tissue in an intended location. One such procedure is VASER® LIPOSELECTION® (hereinafter the “VASER® procedure”). In the VASER® procedure, the doctor makes a small incision in the area to be treated and fills it with a solution containing epinephrine. The doctor then inserts a probe with ultrasonic wave emitting capabilities into the incision. The ultrasonic waves from the probe break down the fat tissue in the targeted area, while leaving the non-targeted tissue substantially intact. Once broken down or otherwise emulsified, a suction device may remove the fat from the targeted area.

Probes utilized in the VASER® procedure have limitations. Two types of probes ordinarily used include a “side cutter” and a “fork tip.” These types of probes are likely to expose the patient to a undue risk of harm. The “side cutter” probe includes an opening on the lateral side of the probe to remove the fat from the patient. The location of the opening therefore restricts the direction of movement of the probe. The side cutter probe must be utilized in a lateral (sideways) movement. Such lateral movement is not recommended during a procedure because it increases the likelihood of torqueing occurring. Torqueing creates friction on or around the area of treatment, which may result in overheating. Overheating is a dangerous condition that can lead to damage to the patient, often times resulting in skin burns.

In the “fork tip” probe, the patient is exposed another type of harm. Fork tip probes include an opening at the distal end of the probe that approximates the shape of a fork or a “V”. To avoid torqueing, the probe is reciprocally moved, rather than in a sideways pattern. During a surgical procedure, however, the integrity of a fork tip probe may be compromised exposing the patient to an increased risk of harm. The tip of the probe forming the “V” can break resulting in pieces of the probe being left inside the patient. Such pieces often times need to be extracted in a subsequent surgical procedure.

Accordingly, there exists a need for an ultrasonic cutting probe that can avoid the limitations of previous probes, and obtain beneficial outcomes in fat removal procedures.

### SUMMARY OF THE INVENTION

The present invention is directed to an ultrasonic cutting probe for use in surgical procedures associated with the removal of fat tissue. The inventive probe is intended to be used as a device that may be inserted into an incision and thereafter be movably and/or removably positioned at an intended location. As such, the inventive probe is structured to transmit ultrasonic energy to an intended location within a surgical site. The inventive probe may be attached to a handle and/or an ultrasonic energy generator or machine.

The probe according to the present invention generally comprises an elongated body having a sidewall that extends between a proximal end and a distal end of the probe, and at least one projection which is structured to transmit ultrasonic energy to the intended location, such as within the surgical site. The sidewall generally comprises a substantially curved transverse sectional configuration, although other transverse sectional configurations are also possible. The projection(s) is disposed on the sidewall, generally in a spaced relation to both the distal end and the proximal end. The projection(s) may have a variety of configurations including, but not limited to, a substantially curved configuration. The projection(s) may extend transversely substantially around the entire perimeter of the sidewall, or around a portion thereof. A projection that extends substantially around entire perimeter of the sidewall at least partially enables fat tissue removal substantially around all sides of the probe, whereas a projection that only partially extends around the perimeter of the sidewall permits a user to more precisely remove fat tissue on an intended lateral location(s) of the surgical site.

The probe according to the present invention further comprises a distal tip, generally disposed between the distal end and the projection(s). The distal tip generally comprises a substantially conical configuration that facilitates entry and/or movable and removable positioning of the probe within the surgical site. This is advantageous at location(s) of the surgical site that may be relatively small or that may otherwise be difficult to access. The configuration of the distal tip also permits a user to gradually insert the probe without causing an abrupt change in pressure. The distal tip generally comprises an outer surface that generally diverges, continuously and outwardly, from the distal end to the projection(s). Said differently, the transverse sectional area of the distal tip continuously increases from the distal end of the probe to the projection(s). The distal tip further comprises an apex having a substantially rounded, blunt configuration. The apex is substantially coincident with the distal end of the probe so that the conical configuration of the distal tip continuously enlarges or increases from the apex to the projection(s).

Additional features of the probe according to the present invention comprise a distal tip with an increased transverse sectional area that at least partially facilitates fat tissue removal. The transverse sectional configuration of the distal tip may diverge outwardly from the distal end, continually increasing to an outer periphery of the projection. At the outer periphery, the transverse sectional area of the distal tip is generally greater than the transverse sectional area of the sidewall. At the point of intersection with the outer periphery, the distal tip may have a substantially elliptical configuration.

Even additional features of the probe according to the present invention comprise a plurality of projections being disposed on the sidewall. Two or more projections may be disposed on the sidewall in a spaced relation to one another, and collectively in a spaced relation to both the proximal and distal ends of the probe. The projections may extend substantially around the entire perimeter of the sidewall, or around a portion thereof. Alternatively, the probe may comprise a combination of a projection(s) that extend substantially around the entire perimeter of the sidewall, and a projection(s) that extend substantially around less than the entire perimeter of the sidewall.

Further features of the present invention include a connecting structure or connector disposed on the proximal end of the probe. The connecting structure may be used to connect the probe to at least one exterior ultrasonic device or generator. The exterior ultrasonic device or generator may comprise a handle for the user to hold and/or position the probe during a surgical procedure.

### BRIEF DESCRIPTION OF THE DRAWINGS

For a fuller understanding of the nature of the present invention, reference should be had to the following detailed description taken in connection with the accompanying drawings in which:

FIG. 1A is a side view of a portion of one embodiment of the ultrasonic probe according to the present invention comprising a projection that extends substantially along the entirety of the sidewall.

FIG. 1B is a front view of the embodiment as represented in FIG. 1A.

FIG. 1C is a perspective view of the embodiment as represented in FIGS. 1A and 1B.

FIG. 2A is a side view of a portion of another embodiment of the ultrasonic probe according to the present invention comprising a projection that extends substantially along a portion of the sidewall.

FIG. 2B is a front view of the embodiment as represented in FIG. 2A.

FIG. 2C is a perspective view of the embodiment as represented in FIGS. 2A and 2B.

FIG. 3A is a side view of a portion of another embodiment of the ultrasonic probe according to the present invention comprising a distal tip having an increased transverse sectional area.

FIG. 3B is a front view of the embodiment as represented in FIG. 3A.

FIG. 3C is a perspective view of the embodiment as represented in FIGS. 3A and 3B.

FIG. 4A is a side view of a portion of another embodiment of the ultrasonic probe according to the present invention comprising two projections disposed on the sidewall.

FIG. 4B is a perspective view of the embodiment as represented in FIG. 4A.

Like reference numerals refer to like parts throughout the several views of the drawings.

### DETAILED DESCRIPTION

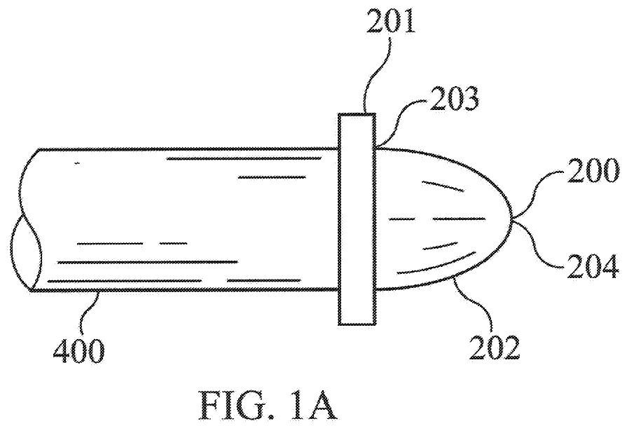

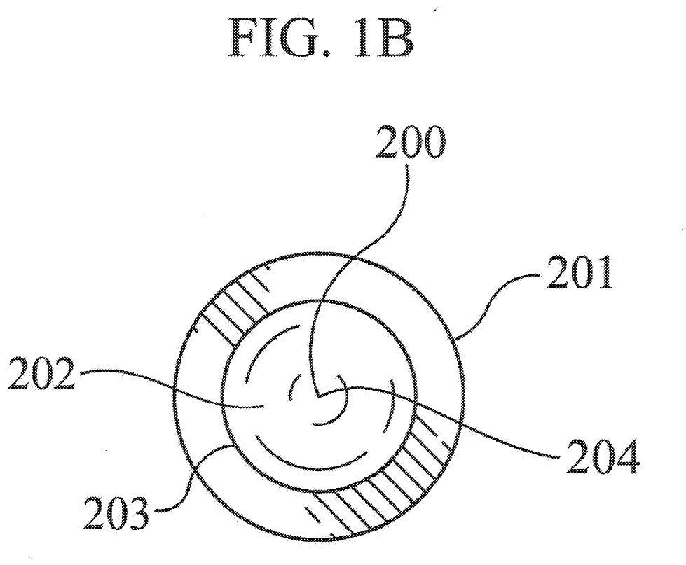

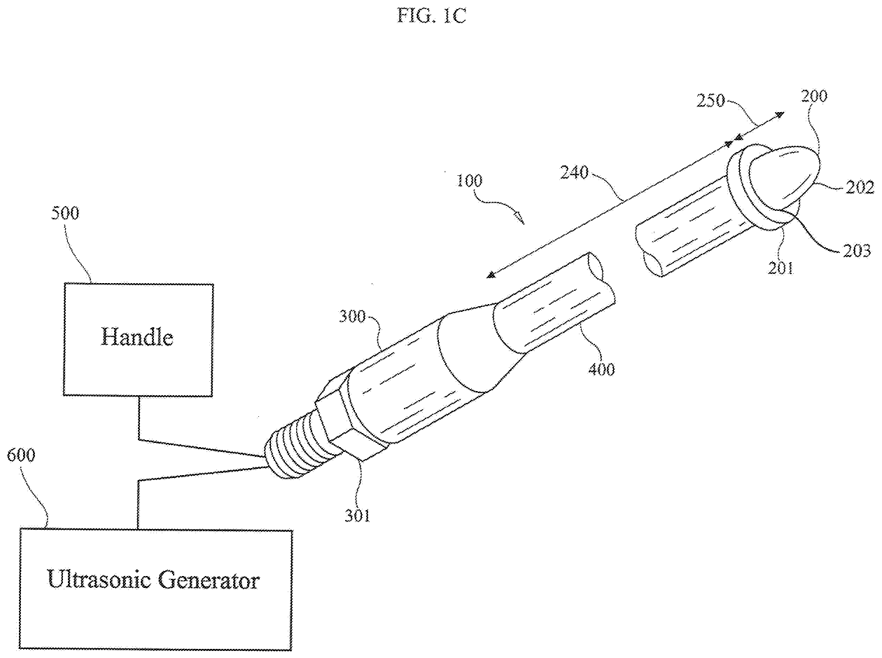

The present invention is directed to an ultrasonic cutting probe for use in fat removal procedures, indicated in FIGS. 1A-4B as 100, which is structured to conduct ultrasonic energy to an intended surgical site. For ease of reference, the ultrasonic cutting probe 100 is referred herein as probe 100. The surgical site may be an area within the body of a patient where fat tissue may accumulate. Such area may be, but is not necessarily limited to, the abdomen, glutes, chest, or a different location within the body of the patient. It is also within the scope of the present invention that the probe 100 be inserted into the body of a patient though an incision, and that it be movably and/or removably positioned at a desired or intended location within the surgical site. With reference to FIG. 1C, it is within the scope of the present invention that the probe 100 be attachable to a handle, which is indicated as 500. As is also shown in FIG. 1C, the probe 100 is also attachable to an ultrasonic generator 600 capable of providing ultrasonic energy to the probe 100. The ultrasonic generator 600 may comprise a handle 500, or alternatively both the handle 500 and the generator 600 may be separate components.

With reference to FIGS. 1A-4B, the inventive probe 100 comprises an elongated body with a sidewall 400 that extends between a proximal end 300 and a distal end 200 of the probe 100. As shown in the illustrative examples of FIGS. 1A-4B, the sidewall 400 may comprise a substantially curved, transverse sectional configuration. However, other transverse sectional configurations of the sidewall 400 are also possible and include, but are not limited to, substantially square, rectangular, hexagonal, heptagonal, octagonal, nonagonal, or other related transverse sectional configurations. The probe 100 according to the present invention further comprises at least one projection, indicated as 201 or 201′, which is intended to transmit ultrasonic energy to the intended surgical site. As is shown throughout the Figures, the projection 201 or 201′ is disposed on the sidewall 400, generally in a spaced relation to the distal end 200, and also in a spaced relation to the proximal end 300. The projection 201 or 201′ is intended to transmit ultrasonic energy to the intended surgical site in a direction that is substantially parallel to a reciprocal direction of movement of the probe 100. The probe 100 is intended to reciprocally move along its own length, such as from the projection 201 or 201′ towards the distal end 200, and/or from the projection 201 or 201′ and towards the proximal end 300.

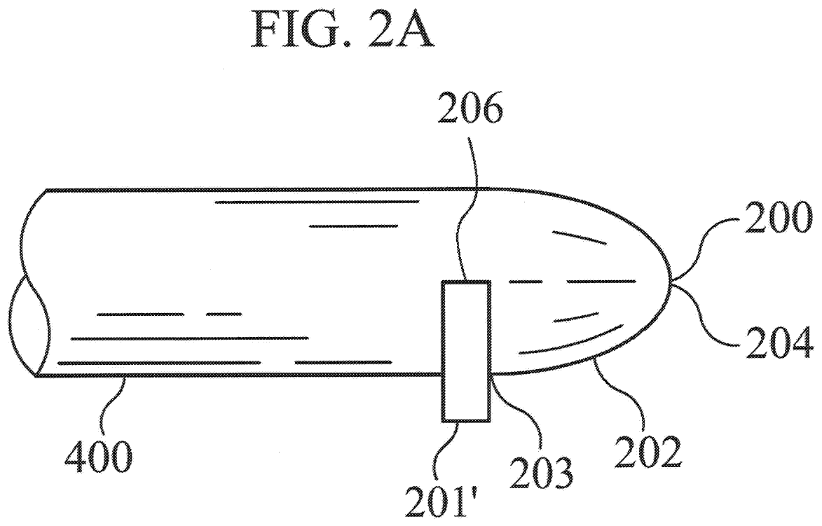

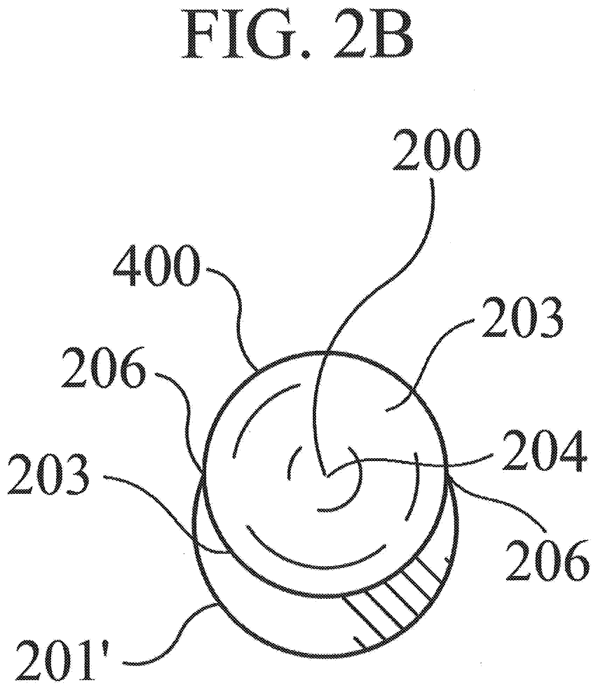

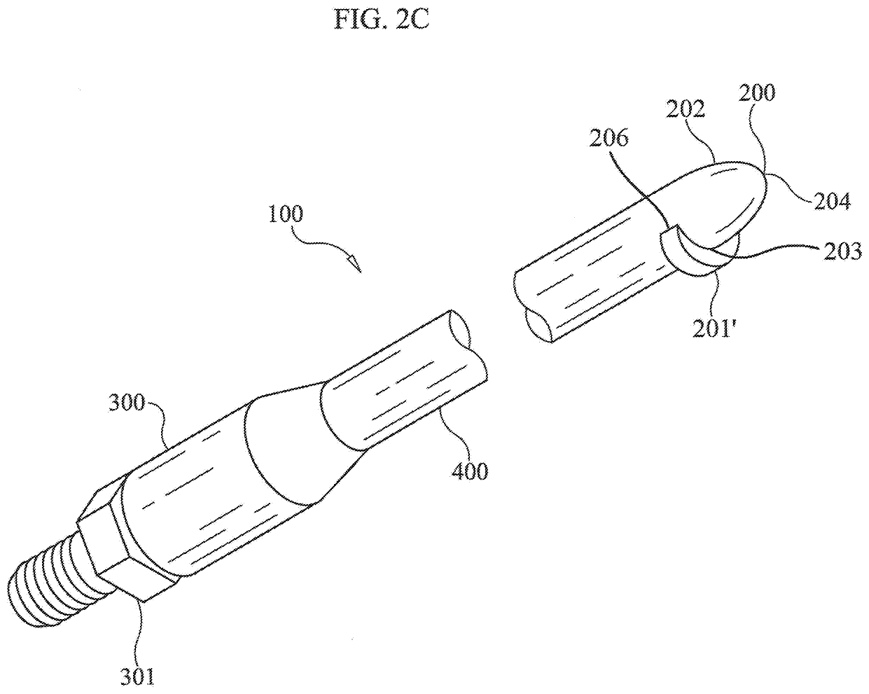

As may be best appreciated in FIGS. 1B, 2B, and 3B, the projection 201 or 201′ may have a substantially curved configuration. The configuration of the projection 201 or 201′ may be circular, semi-circular, elliptical, or another related curved configuration. As may also be appreciated at least from FIGS. 1B, 2B, and 3B, the projection 201 or 201′ is disposed on the sidewall 400, and generally extends transversely, and at least partially, around the perimeter of the sidewall 400. As used herein, and as shown in FIGS. 1A-1C, a projection indicated as 201, extends transversely around substantially the entire perimeter of the sidewall 400. This at least partially facilitates removal of fat tissue substantially around all sides of the probe 100, which may at least partially reduce the overall time associated with the underlying surgical procedure. Conversely, in other instances it may be advantageous to provide a probe 100 that comprises a projection, indicated as 201′, which extends transversely substantially around less than the entire perimeter of the sidewall 400. Such a projection 201′ generally permits a user to more precisely remove fat tissue at an intended lateral location(s) of the surgical site. The configuration of the projection 201′, which extends substantially around less than the entire perimeter of the sidewall 400, may also at least partially reduce unintended damage to certain locations of the surgical site when positioning the probe 100. Further, in the embodiments as represented in FIGS. 2A-4B, the projection(s) 201′ smoothly transitions into the sidewall 400 at an intersection, indicated as 206. Other configurations are also possible and include the projection(s) 201′ transitioning into the sidewall 400 in a substantially perpendicular alignment or orientation relative to the sidewall 400.

As may be appreciated from FIGS. 1A-4B, the probe 100 according to the present invention comprises a distal tip 202 that is generally disposed between the distal end 200 of the probe 100 and the projection 201 or 201′. The distal tip 202 generally comprises a substantially conical configuration, which facilitates movable and/or removable positioning of the probe 100, such as within a relatively small intended location(s) of the surgical site. Furthermore, with a substantially conical configuration of the distal tip 202, the probe 100 may be gradually inserted into a relatively small intended location(s) of the surgical site without causing an abrupt increase in pressure. The distal tip 202 generally comprises an outer surface that generally diverges, continuously and outwardly, from the distal end 200 and to the projection 201 or 201′. In embodiments of the probe 100 comprising more than one projection 201 or 201′, the distal tip 202 generally diverges continuously and outwardly, from the distal end 200 to a first adjacent one of the projections 201 or 201′. Therefore, the transverse sectional configuration, and consequently the transverse sectional area, of the distal tip 202 continuously increases from the distal end 200 to the projection 201 or 201′. In the illustrative embodiments as represented in FIGS. 1A-4B, the distal tip 202 comprises a continuously increasing or enlarging circular transverse sectional configuration.

As may also be appreciated from the Figures, the distal tip 202 generally comprises an apex, indicated as 204, which is substantially coincident with the distal end 200. The apex 204 generally comprises a substantially rounded, blunt configuration, which facilitates positioning of the probe 100. Thus, the substantially conical configuration of the distal tip 202 may further comprise a continuously enlarging circular transverse sectional configuration that extends from the apex 204 to the projection 201 or 201′. As represented in the illustrative embodiment of FIG. 1A, the distal tip 202 diverges continuously from the distal end 200, such as around apex 204, and to a base 203 of the projection 201. At the base 203, the transverse sectional area of the tip 202 is roughly equivalent to the transverse sectional area of the rest of the sidewall 400. As may be appreciated from FIG. 1C, a first length 240 corresponds to the length of the distal tip 202, and is defined as the distance between the apex 204, which is substantially coincident to the distal end 200, and the projection 201 or 201′. As may be also appreciated from FIG. 1C, a second length 250 corresponds to the length of the sidewall 400, and is roughly defined as the distance between the projection 201 or 201′ and the proximal end 300. The first length 240 is generally shorter than the second length 250.

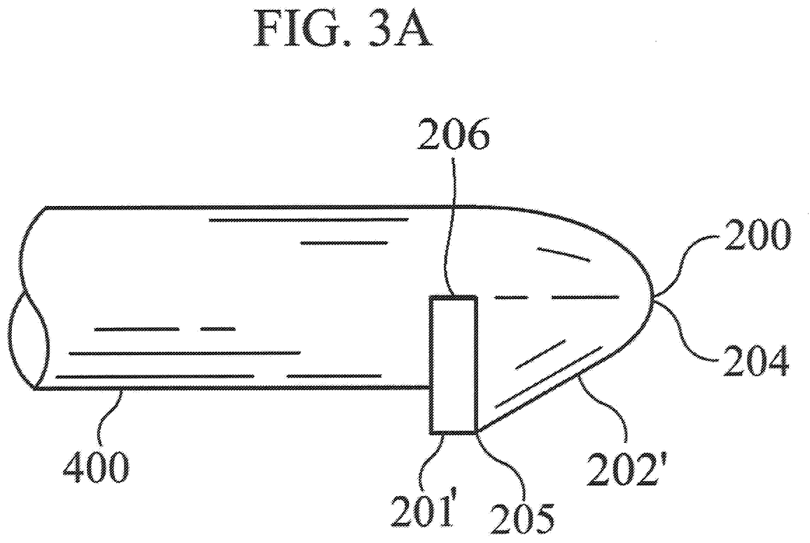

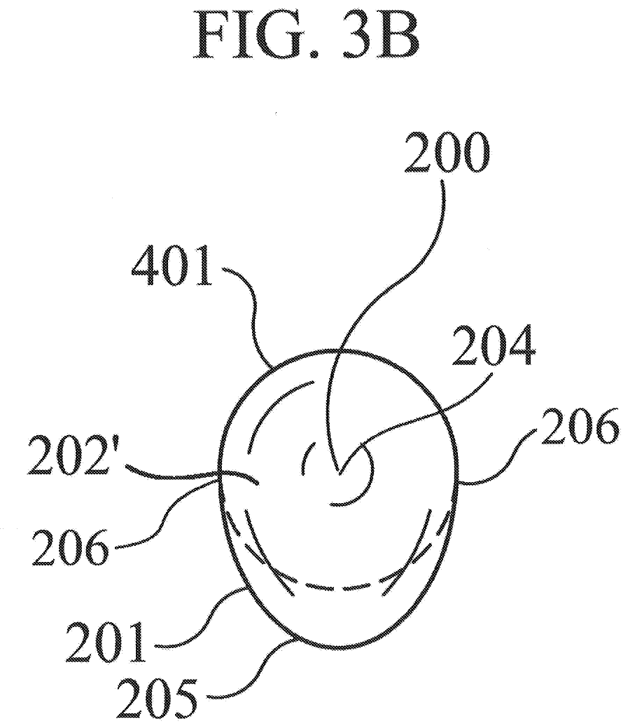

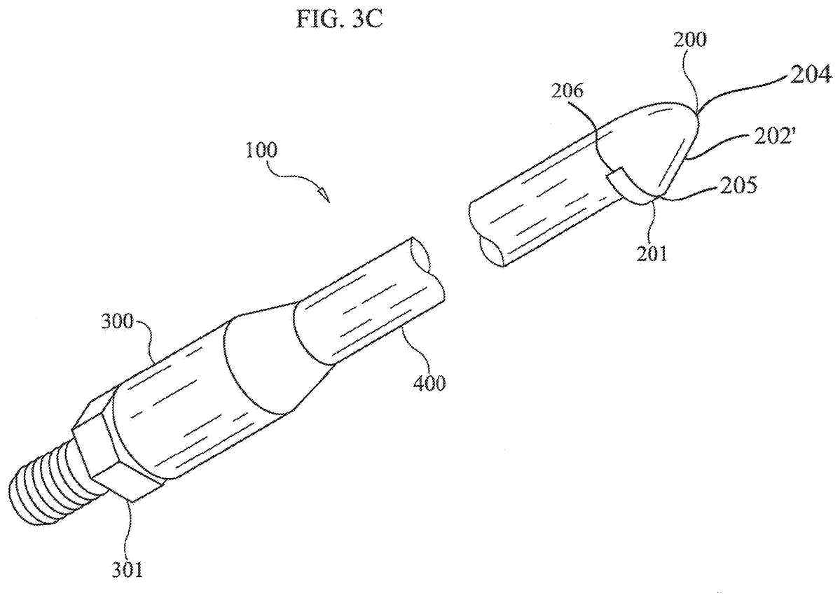

As shown in the illustrative embodiment of FIGS. 3A-3C, additional features of the present invention comprise providing a probe 100 having a distal tip 202 with an increased transverse sectional area. An increased transverse sectional area of the distal tip 202 results in an increased area of the surface of the tip 202, which may at least partially enhance fat tissue removal. As shown in FIGS. 3A-3C, the transverse sectional area of the distal tip 202 diverges outward from the distal end 200, such as from the apex 204, and continually increases to an outer periphery 205 of the projection 201′, at which point the transverse sectional area of the distal tip 202 is greater than the transverse sectional area of the sidewall 400. As may be appreciated from the illustrative embodiment of FIG. 3B, the outer periphery 205 may have a substantially elliptical configuration, wherein approximately half of the outer periphery is substantially coincident with the perimeter of the sidewall 400. Other configurations are also possible. For example, the outer periphery 205 need not be substantially coincident with the perimeter of the sidewall 400, or with a portion thereof. Instead, the transverse sectional area of the tip 202 at the outer periphery 205 may be greater than the transverse sectional area of the sidewall 400.

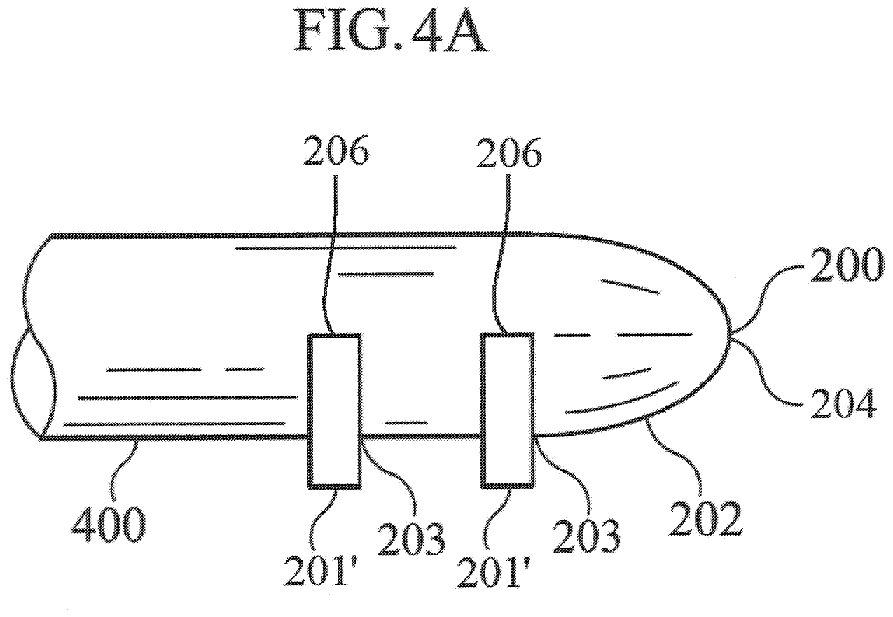

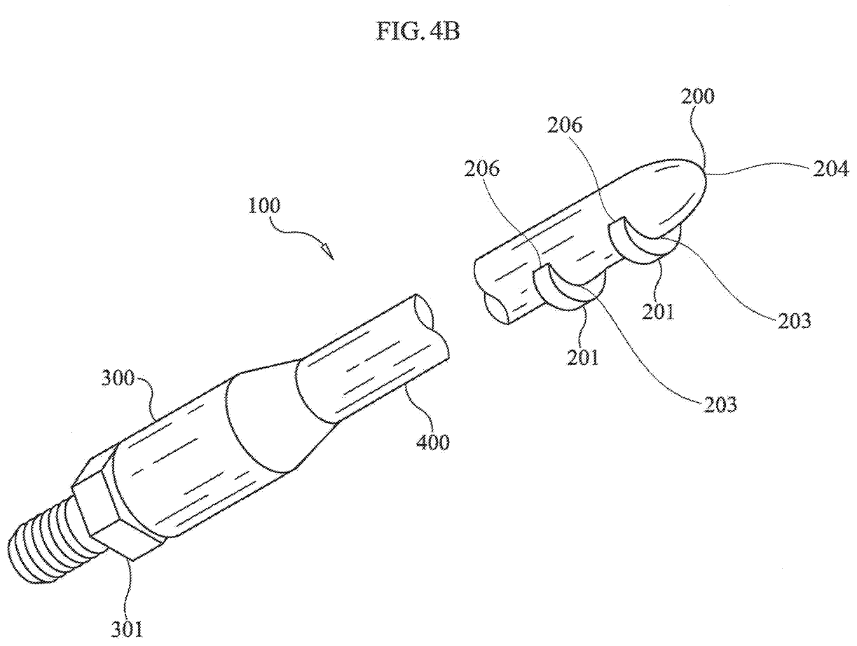

As represented in FIGS. 4A and 4B, additional features of the probe 100 according to the present invention comprise more than one projection, such as 201 or 201′, disposed along the sidewall 204. As shown in FIGS. 4A and 4B, the projections 201 or 201′ may be disposed on the sidewall 400 in a spaced relation to one another. The projections 201 or 201′ are also collectively disposed in a spaced relation to the proximal end 300 and to the distal end 200. In the illustrative embodiments of FIGS. 4A and 4B, two projections 201′ are disposed on the sidewall, each of which has a substantially curved configuration, and each of which extends substantially around one half of the perimeter of the sidewall 400. It is also possible for a probe 100 to comprise a plurality of projections 201, each of which extends substantially around the entirety of the perimeter of the sidewall 400. Other configurations are also possible and include combinations of projection(s) 201 and 201′ disposed on the sidewall 400.

As is shown in FIGS. 1C, 2C, 3C, and 4B, the probe 100 according to the present invention may comprise a connecting structure or connector 301 which may be used to connect the probe 100 to at least one exterior ultrasonic device or generator, which is indicated as 600 in FIG. 1C. The exterior ultrasonic device or generator 600 may be a handle, which the operator may hold during a surgical procedure to position the probe 100. When used in connection with a handle, the probe 100 is generally connected to the handle 500 prior to connecting the probe with an ultrasonic generator 600.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A probe attachable to an ultrasonic generator and structured to conduct ultrasonic energy to a surgical site, said probe comprising:
an elongated body having a distal end and a proximal end,
said body including a sidewall extending between said distal and proximal ends,
at least one projection extending outwardly from said sidewall and structured to remove fat tissue from the surgical site,
said body further including a distal tip disposed between said distal end and said at least one projection,
said at least one projection having a curved configuration extending transversely and at least partially around said sidewall, and
said distal tip and said projection structured and dimensioned for movable and removable positioning within the surgical site.

2. The probe as recited in claim 1 wherein said at least one projection extends transversely around an entirety of said sidewall.

3. The probe as recited in claim 1 wherein said at least one projection extends transversely around at least a majority of said sidewall.

4. The probe as recited in claim 1 wherein said at least one projection extends transversely around substantially one half of said sidewall.

5. The probe as recited in claim 1 further comprising a plurality of projections each extending outwardly from said sidewall and structured to remove fat tissue from the surgical site.

6. The probe as recited in claim 5 wherein each of said plurality of projections includes a curved configuration extending transversely and at least partially around said sidewall.

7. The probe as recited in claim 6 wherein said distal tip is disposed between said distal end and said plurality of projections collectively.

8. The probe as recited in claim 7 wherein said distal tip and said plurality of projections are collectively dimensioned and structured for movable and removable positioning within the surgical site.

9. The probe as recited in claim 6 wherein said distal tip and said plurality of projections are collectively dimensioned and structured for movable and removable positioning within said surgical site.

10. The probe as recited in claim 1 wherein said distal tip comprises a substantially conical configuration having an outer surface diverging continuously outward from said distal end to said at least one projection.

11. The probe as recited in claim 10 wherein said substantially conical configuration comprises an apex disposed coincident with said distal end; said apex comprising a substantially rounded, blunt configuration.

12. The probe as recited in claim 11 wherein said substantially conical configuration further comprises a continuously enlarging circular transverse sectional configuration extending from said apex to said at least one projection.

13. The probe as recited in claim 12 wherein said outer surface of said distal tip diverges continuously outward from said apex to a base of said at least one projection.

14. The probe as recited in claim 10 wherein said outer surface of said distal tip diverges continuously outward from said distal end to a base of said at least one projection.

15. The probe as recited in claim 10 wherein said outer surface of said distal tip diverges continuously outward from said distal end to an outer periphery of said at least one projection.

16. The probe as recited in claim 15 wherein said at least one projection extends around substantially one half of said sidewall.

17. The probe as recited in claim 16 wherein said substantially conical configuration comprises an apex coincident with said distal end; said apex comprising a substantially rounded, blunt configuration.

18. The probe as recited in claim 1 wherein said at least one projection is disposed on said sidewall in a spaced relation to said distal end and said proximal end.

19. The probe as recited in claim 1 wherein said distal tip has a length shorter than that of a remainder of said sidewall extending between said at least one projection and said proximal end.

20. The probe as recited in claim 1 further comprising a connector structured for attachment to a handle.

21. A probe attachable to an ultrasonic generator and structured to conduct ultrasonic energy to a surgical site, said probe comprising:
an elongated body having a distal end and a proximal end,
said body including a sidewall extending between said distal and proximal ends,
at least one projection extending outwardly from said sidewall and structured to remove fat tissue from the surgical site,
said body further including a distal tip disposed between said distal end and said at least one projection,
said distal tip comprising a substantially conical configuration diverging continuously from said distal end to said at least one projection,
said substantially conical configuration comprising an apex disposed coincident to said distal end, said apex comprising a substantially rounded, blunt configuration,
said at least one projection comprising a curved configuration and extending transversely and at least partially around said sidewall,
said distal tip comprising a length shorter than that of a remainder of said sidewall extending between said at least one projection and said proximal end, and
said distal tip and said projection structured and dimensioned for movable and removable positioning within the surgical site.
